(19) United States
(12) Patent Application Publication
(10) Pub. No.: US 2017/0095615 A1
(43) Pub. Date: Apr. 6, 2017

(54) WEIGHT-BASED DOSING SYRINGE

(71) Applicants: Gwenyth Anne Fischer, Minneapolis, MN (US); Laura-Lee Farrell Brown, Maplewood, MN (US); Brian Mullins, Minneapolis, MN (US); Braden Jeffery Eliason, Minneapolis, MN (US); Alan Lloyd Carlson, St. Paul, MN (US)

(72) Inventors: Gwenyth Anne Fischer, Minneapolis, MN (US); Laura-Lee Farrell Brown, Maplewood, MN (US); Brian Mullins, Minneapolis, MN (US); Braden Jeffery Eliason, Minneapolis, MN (US); Alan Lloyd Carlson, St. Paul, MN (US)

(21) Appl. No.: 15/282,853

(22) Filed: Sep. 30, 2016

Related U.S. Application Data
(60) Provisional application No. 62/236,443, filed on Oct. 2, 2015.

(51) Int. Cl. A61M 5/315

(52) U.S. Cl. CPC A61M 5/31556; A61M 5/31563; A61M 2005/3126

(57) ABSTRACT

A system includes a syringe barrel, a syringe plunger, and an annular collar. The syringe barrel has a longitudinal axis aligned axially between a discharge end and a cylinder end. The syringe plunger has a cap disposed on a first end, a piston disposed on a second end and configured for disposition in a bore of the barrel, and has a body disposed between the first end and the second end. The body has an external thread on a first surface and the body has a visible calibration indication aligned axially. The annular collar has at least one interrupted internal thread segment on an interior surface. The collar has elasticity such that in a first state, the internal thread is configured to engage the external thread and in a second state, in which a compressive force is exerted on opposing sides of the collar, the internal thread is disengaged from the external thread.

### CLAIM OF PRIORITY

This patent application claims the benefit of priority of U.S. Provisional Patent Application Ser. No. 62/236,443, filed on Oct. 2, 2015, which is hereby incorporated by reference herein in its entirety.

### BACKGROUND

Syringe-based vaccinations are commonly used for treating medical conditions. For example, and under certain conditions, the standard care for an adult patient having a cardiac arrest entails a dose of epinephrine.

### OVERVIEW

For an adult patient, epinephrine is readily available in a pre-packaged injectable dose. For pediatric patients, the dosing is weight-based and the particular volume must be individually calculated and prepared. The dosing preparation work for a pediatric patient increases the delay in providing treatment to the patient, and in some instances, this delay can lead to substantial medical complications.

Multiple steps including performing mathematical calculations in combination with the urgency of the situation and unfamiliarity of most providers with resuscitating pediatric cardiac arrest create a scenario prone to errors. According to some researchers, up to 50%-60% of the time epinephrine is administered during a pediatric cardiac arrest is incorrectly dosed. In addition to errors in dosing, the multi-step preparation process causes delays in delivery that may increase the risk of mortality.

One example of the present subject matter includes a pre-filled single dose syringe with accurate kilogram weight markings for use in pediatric populations. The weight markings can be indicated on the side of the syringe barrel, the plunger, or on another conspicuous surface. The syringe can be fabricated of plastic, glass, or a polymer. Some examples of suitable polymers include cyclic olefin copolymer (COC), cyclo-olefin polymers (COP), and cationic conjugated polymer (CCP).

One example includes a circumferential ring-shaped stopper configured to slide on the syringe plunger. The stopper position can be manipulated by the user and the position can be selected according to a patient weight. The stopper is sometimes called a collar.

In one example, the calibration markings on the syringe correspond with patient weight and align with a standard color marking scheme. One such color marking scheme is known as the Broselow color markings. For example, a Broselow Pediatric Emergency Tape correlates patient weight with a color system for pediatric emergencies.

One example of a color system includes specific colors associated with a particular weight (expressed in kilograms or pounds) and one example is shown in the accompanying table. In the various examples disclosed herein, the calibration markings can be screen-printed or formed by embossed or raised markings.

| | Weight | Weight |
|---|---|---|
| Color | (kilograms) | (pounds) |
| Grey | 3-5 | 6-11 |
| Pink | 6-7 | 13-15 |
| Red | 8-9 | 17-20 |
| Purple | 10-11 | 22-24 |
| Yellow | 12-14 | 26-30 |
| White | 15-18 | 33-40 |
| Blue | 19-23 | 42-50 |
| Orange | 24-29 | 53-64 |
| Green | 30-36 | 66-80 |

An example of the present subject matter can be made available for use in an emergency room, an operating room, and an ambulance, and included in a transport kit or a hospital code bag. An example can be operated by a medical provider such as a nurse, certified registered nurse anesthetist, a physician, a physician assistant, a nurse practitioner, a first responder, or an emergency medical technician.

In one example, a device is packaged in a plastic wrapping. Using the device can include removing the plastic wrapping and selecting the patient weight by positioning a ring-shaped stopper. The stopper can be repositioned by sliding or by rotating on threads. A removable safety cap on the syringe needle tip can reduce the incidence of accidental ‘sticks.’ The user can expel air from the tip in a manner similar to other syringes and can attach the syringe to other devices using a quick connect fitting such as a Luer-lock system (to an IV) or an Intraosseous access coupled to the patient. The user then manipulates the syringe plunger to deliver the dose. The plunger movement is limited, or stopped, by the ring-shaped stopper at a position corresponding to the appropriate dose based on the patient weight. The dose can include epinephrine or other weight-based medicament. In one example, the patient age is indicated on a gradations or calibration markings on the syringe and dosing is determined based on patient age indicated by a color, by years, or months.

The present inventors have recognized, among other things, that a problem to be solved can include rapidly determining proper dosing for a syringe-based medicament. The present subject matter can help provide a solution to this problem, such as by providing a syringe in which the dose can be selected based directly on patient weight and without need for calculations, transformation, or data manipulations. In one example, selecting the dose entails positioning a mechanical feature of the syringe in a manner that restricts a deliverable volume of medicament.

Each of these non-limiting examples can stand on its own, or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 includes a view of a syringe assembly according to one example.

FIG. 2 includes a view of a collar according to one example.

FIGS. 3A and 3B include views of plunger bodies.

FIG. 4 includes a view of a syringe assembly according to one example.

FIGS. 5A and 5B include views of a collar according to one example.

FIG. 6A includes a view of a plunger body and collar according to one example.

FIG. 6B includes a view of a collar according to one example.

FIG. 7 includes a view of a syringe assembly according to one example.

FIG. 8 includes a view of a bespoke coupler according to one example.

FIG. 9 includes a view of a syringe assembly according to one example.

FIG. 10 includes a view of a syringe assembly according to one example.

FIG. 11 includes a view of a syringe assembly according to one example.

FIG. 12 includes a view of a syringe assembly according to one example.

FIG. 13 includes a view of a syringe assembly according to one example.

FIG. 14 includes a flow chart of a method according to one example.

### DETAILED DESCRIPTION

This document discloses weight-based syringes configured for single dose use.

By reducing epinephrine dosing errors, for example, during a pediatric arrest, the present subject matter can decrease the morbidity and mortality associated with certain medical conditions.

One example includes a ring-shaped stopper that is configured to slide or screw along an axis of the plunger. In one example, weight markings, such as kilograms, are conspicuously displayed on the side of the syringe. For example, the Broselow color system can be provided on the syringe.

In addition, to epinephrine, other examples of metered dosing is also contemplated. For example, with a pulmonary processes, the medicament can include an inhaled asthma medication. In addition, the medicament can include seizure medications such as Ativan, Amiodarone (for arrhythmias), calcium or other electrolytes.

An example can include an auto-injector for pediatric cardiac arrest. The auto-injector can be configured for delivering a pre-filled adult dose and can be configured for variable dosing at a level suitable for pediatric use. Configuring can include labelling or can include providing a user-positionable mechanical depth-stop component that limits the dose delivery.

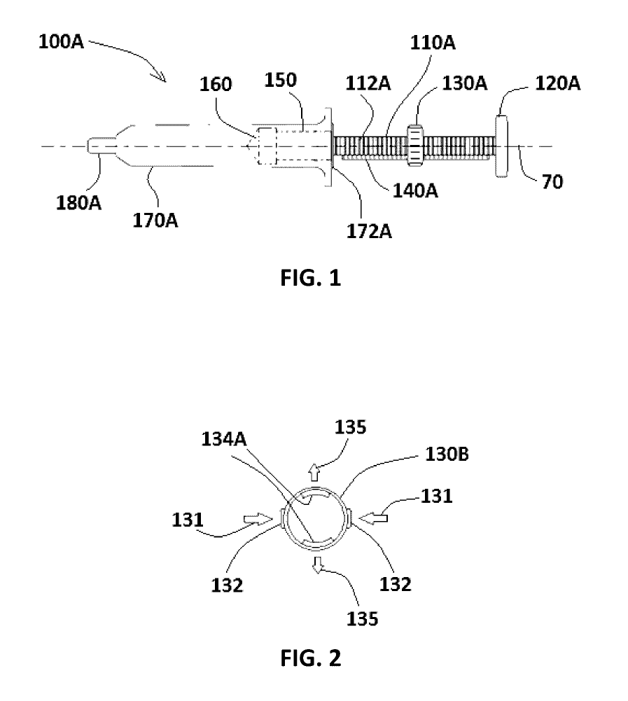

FIG. 1 includes a view of syringe 100A, according to one example. Syringe 100A includes barrel 170A having a discharge port associated with connector 180A and an opening end associated with a surface 172A. Barrel 170A has an internal bore aligned on longitudinal axis 70 and in which piston 160 travels. Connector 180A can include a Luer-lock connector to accept a needle or other connector to enable coupling with medical equipment.

Plunger 110A terminates at a first end at piston 160 and at a second end at cap 120A. Between piston 160 and cap 120A, plunger 110A includes a plunger body. The plunger body includes threaded portion 112A, scale 140A, and, in the example shown, segment 150. Piston 160 can include a rubber or elastic material to ensure a fluid-tight seal with the internal bore of barrel 170A. Threaded portion 112A in the example shown, includes interrupted threads in which a section of thread along the longitudinal axis 70 has been removed. Scale 140A, in the example shown, includes a raised flange having gradations or markings that correlate with a medicament volume. Segment 150 provides a substantially rigid coupling between piston 160 and threaded portion 112A. Threaded portion 112A can fully encircle the diameter or partially encircle the diameter.

Collar 130A is a ring-shaped element having internal threads corresponding to threaded portion 112A and having an external surface configured for manual manipulation. A position of collar 130A relative to plunger 110A is user-selectable. Collar 130A can be repositioned by two methods. In a first method, collar 130A can be manually distorted so that the internal threads disengage from the threaded portion 112A and thus allow the collar to slide along the threaded portion 112A in the direction aligned with axis 70. In a second method, collar 130A can be rotated about axis 70 and the alignment of the internal threads and threaded portion 112A will drive collar 130A along the length of plunger 110A.

Scale 140A can include markings to indicate patient chronological age, patient weight, or dosing volume. The markings can include a color scale.

Syringe 100A can be fabricated of various materials, including plastic, glass, or a polymer.

FIG. 2 includes a view of collar 130B according to one example. The ring-shaped configuration of collar 130B is shown in the figure. In the example shown, collar 130B includes two segments of internal threads 134A disposed on diametrically opposing regions of the interior surface. In addition, collar 130B includes finger pads 132 on an exterior surface and disposed on diametrically opposing regions. The arrangement of the finger contact pads 132 are substantially perpendicular to the arrangement of the internal threads 134A.

A compressive force exerted by fingers of a user and applied to the finger pads 132, exerted in the directions indicated by arrows 131, will cause distortion of the shape of collar 130B. The resulting distortion will cause movement of internal threads 134A in the direction indicated by arrows 135. Movement of internal threads 134A in the direction indicated will cause disengagement with threaded portion 112A and permit collar 130B to be moved in direction aligned with axis 70. Collar 130B can be fabricated of a plastic or polymer material. Collar 130B can be described as having a first state in which the threads are engaged and having a second state in which the threads are disengaged.

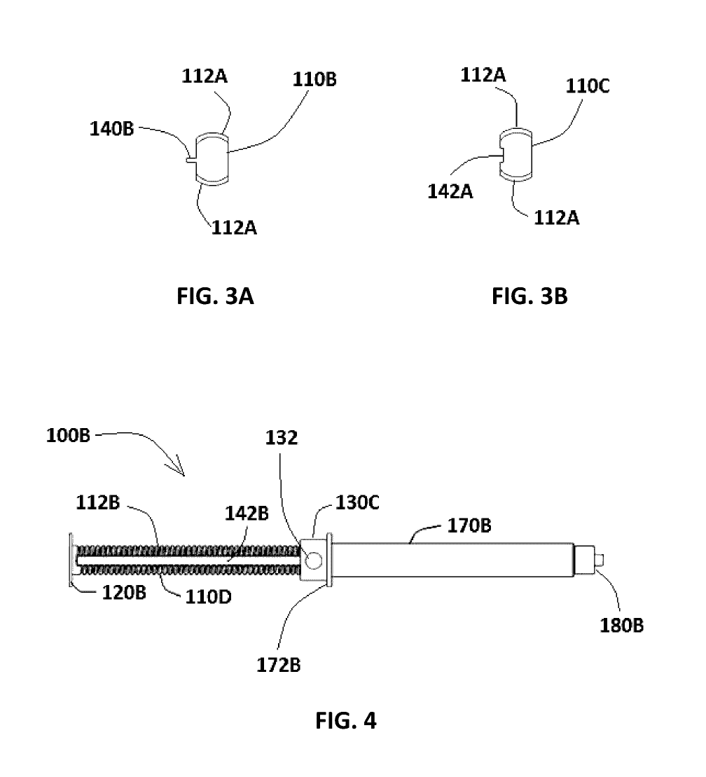

FIGS. 3A and 3B include views illustrating the flatted profile of plunger 110B and 110C. In FIG. 3A, plunger 110B includes threaded portions 112A on curved sides and scale 140B disposed on a flat surface (such as a flange) of plunger 110B. Visible gradation markings can be depicted on a surface of scale 140B. In FIG. 3B, plunger 110C includes threaded portions 112A on curved sides and a scale can be disposed in recessed portion 142A disposed on a flat surface of plunger 110C. Visible gradation markings can be depicted on a surface of recessed portion 142A.

FIG. 4 includes a view of syringe 100B according to one example. Syringe 100B includes barrel 170B fitted with connector 180B at one end and surface 172B at an open end. Plunger 110D includes cap 120B, threaded portion 112B, and a piston (disposed within barrel 170B in this example). Plunger 110D also includes a scale set in recessed portion 142B. Collar 130C is fitted to plunger 110D and is shown positioned such that surface 172B is in contact with collar 130C, thus preventing further ejection of medicament from syringe 100B. Collar 130C includes finger pad 132 in this example.

Barrel 170B is configured for receiving the medicament. The piston within barrel 170B is configured for axial movement within the barrel. Cap 120B can be manipulated by a user to draw or eject medicament.

Syringe 100B includes collar 130C that is repositionably coupled to the barrel. The collar is shown as a ring-shaped component having internal threads that engage with corresponding threads on an external surface of the plunger. In one example, the plunger has markings (such as color-coded markings) and numerical values corresponding to weight values. In this example, the markings are aligned with an edge or surface of the collar and are calibrated to correlate with patient weight. A surface of the collar is configured to limit movement of the cap and thereby limit the deliverable dose. The stopping position is user-selectable by moving the collar 130B. In one example, a course adjustment in the position can be made by manipulating the collar to disengage the threads and a fine adjustment can be made by rotating the collar. The range of movement of the plunger is limited by the position of the collar 130C relative to surface 172B of barrel 170B.

The calibration indications can be depicted in the channel or recessed portion 142B of plunger 110D. In the example shown, the particular markings are omitted for clarity.

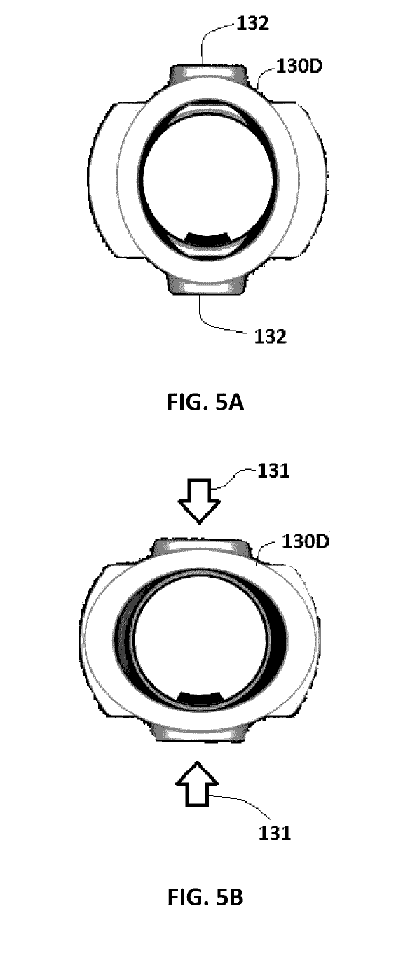

FIGS. 5A and 5B include axial views of collar 130D according to one example. In FIG. 5A, collar 130D is in a first state which can be viewed as a natural or ordinary state. In this state, threads of collar 130D are positioned to engage corresponding threads of plunger 110D. In the view shown, an operative portion of collar 130D has an elliptical shape. The small diameter dimension of the elliptical shape includes internal threads configured to engage corresponding external threads on the plunger (not shown in this view). The larger diameter dimension of the elliptical shape is configured to clear the corresponding external threads on the plunger. The view shown here can be considered an ‘engaged’ mode. The figure illustrates finger pads 132 on collar 130D.

In FIG. 5B, a compressive force is exerted as shown at arrows 131, here shown aligned on opposite sides. The compressive force distorts the shape of operative portion of collar 130D and, as shown, the portion has a circular shape. The diameter dimension of the circular shape is configured to clear the corresponding external threads on the plunger. The view shown here can be considered a ‘disengaged’ mode.

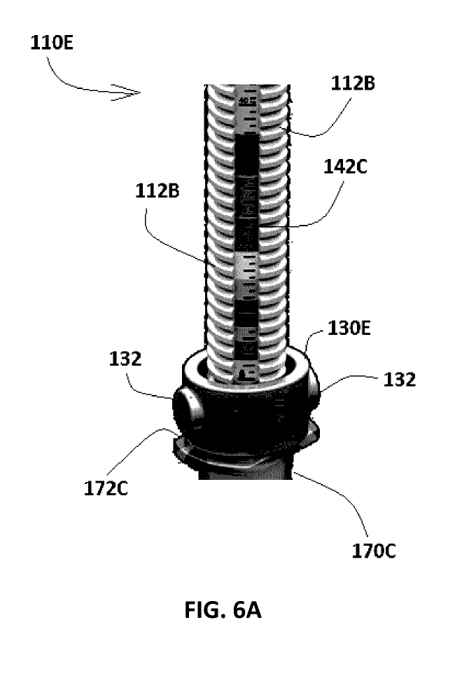

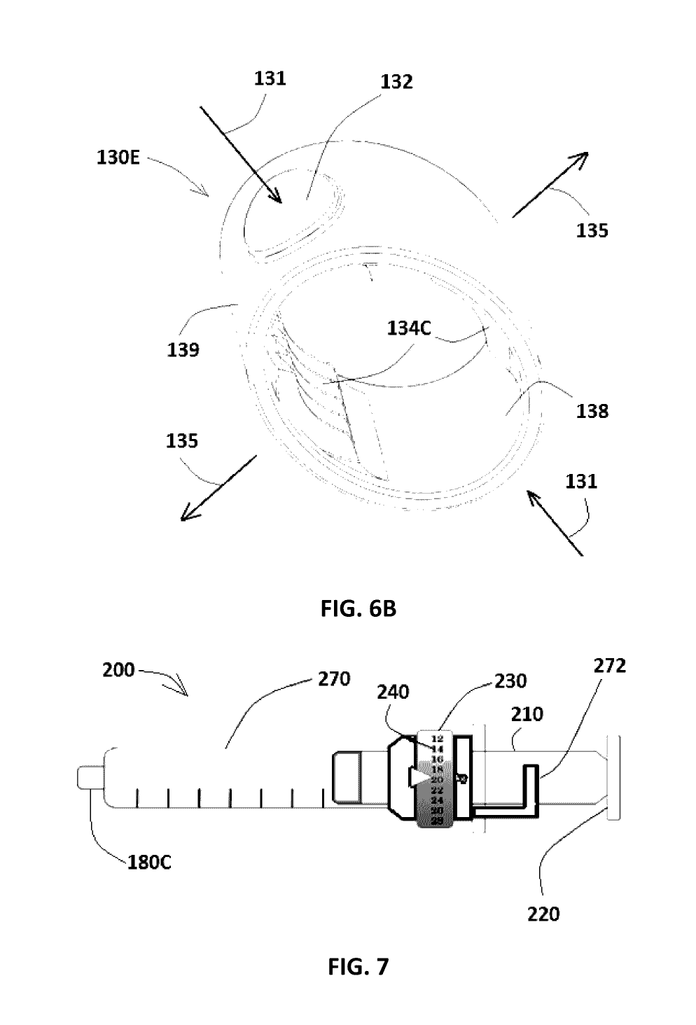

FIG. 6A includes a view of plunger body 110E and collar 130E according to one example. In the figure, calibration markings are indicated in a channel or recessed portion 142C of the plunger body 110E. Plunger body 110E includes external threads 112B configured to engage corresponding internal threads on the bore of collar 130E. Finger grip pads 132 on the external surface of collar 130E correspond to those portions indicated by arrows in FIG. 5B.

A face of collar 130E is shown in a position nearly in contact with face 172C of barrel 170C. When collar 130E is in contact with face 172C, further movement of plunger 110E in a downward direction (as viewed in the figure) is precluded.

FIG. 6B includes a view of collar 130E according to one example. Collar 130E includes an open-ended cylindrical structure having raised finger pads 132 (opposing pad not shown in this view) on opposing sides of external surface 139 and having threads 134C on portions of interior surface 138. Threads 134C are positioned out of phase with finger pads 132. Collar 130E is fabricated of a material that is sufficiently flexible that a user can exert a compressive force on the finger pads 132 (in a direction indicated by arrows 131) and thereby deflect the internal threads 134C (in a direction indicated by the arrows 135) sufficient to disengage from external threads of a corresponding component. When the compressive force is removed, collar 130E returns to a configuration whereby the internal threads 134C are again engaged with the aforementioned external threads.

FIG. 7 includes a view of syringe 200 according to one example. Syringe 200 includes barrel 270 for receiving a medicament. Syringe 200 includes plunger 210 having a piston at a first end and cap 220 at the second end. The piston is configured for axial movement within barrel 270. Cap 220 can be manipulated by a user. Syringe 200 includes collar 230 that is rotatably coupled to barrel 270. Collar 230 has peripheral markings 240 corresponding to weight values. In this example, the markings are aligned with an index mark (triangular feature) calibrated to correlate with patient weight. Collar 230 is coupled to stop 272. The axial position of stop 272 is user-selectable by rotation of collar 230. A surface of the stop limits the travel of plunger 210 and is configured to engage a corresponding surface of cap 220. A discharge end of barrel 270 includes fitting 180C.

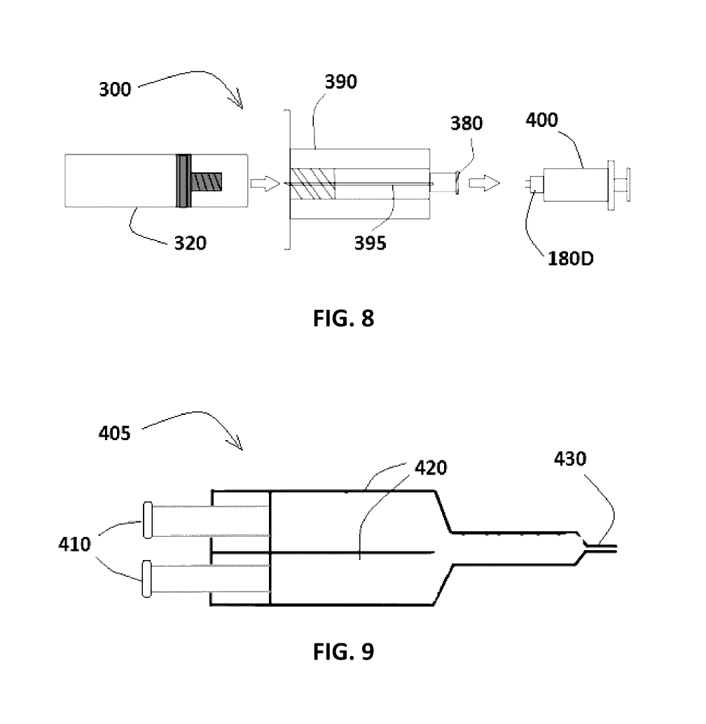

FIG. 8 includes a view of system 300 including bespoke coupler 390, according to one example. Bespoke coupler 390 provides an interface between epinephrine vial 320 and syringe 400. In this example, coupler 390 includes fitting 380 configured to engage a corresponding fitting 180D (such as a Luer-lock) of syringe 400. A needle component 395 of coupler 390 engages vial 320. This configuration averts the need for an adapter to connect a syringe and a vial.

The example in FIG. 8 can be configured for use with an adult-sized auto-injector.

FIG. 9 includes a view of syringe 405 according to one example. Syringe 405 includes dual barrels 420 and dual plungers 410 arranged in parallel alignment. In this example, the barrels (and plungers) can be of different sizes (such as diameter). In different examples, the plungers can be operated independently or as a unit. For example, a first plunger (in a first barrel) can be operated to deliver a first volume and a second plunger (in a second barrel) can be operated to deliver a second volume wherein the total volume is correlated with the patient weight. A discharge end of syringe 405 includes fitting 430.

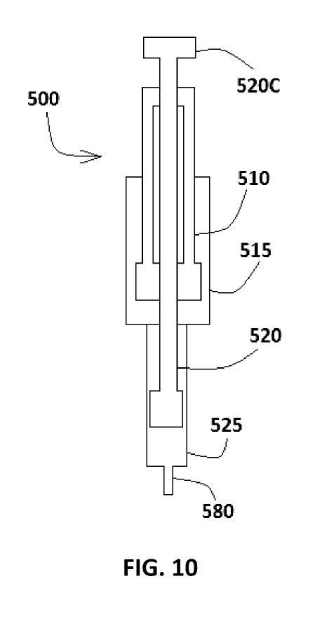

FIG. 10 includes a view of syringe 500 according to one example. Syringe 500 includes a stepped barrel configuration. In this example, the barrels are coaxial (and concentric) and of different dimensions (here, diameter). The example shown includes an inner combination including inner barrel 525 and inner plunger 520 and an outer combination including outer barrel 515 and outer plunger 510. There can be any number of separate combinations and the combinations can be selectively deployed to deliver a selected dose. Actuation of cap 520C causes ejection of medicament via connector 580.

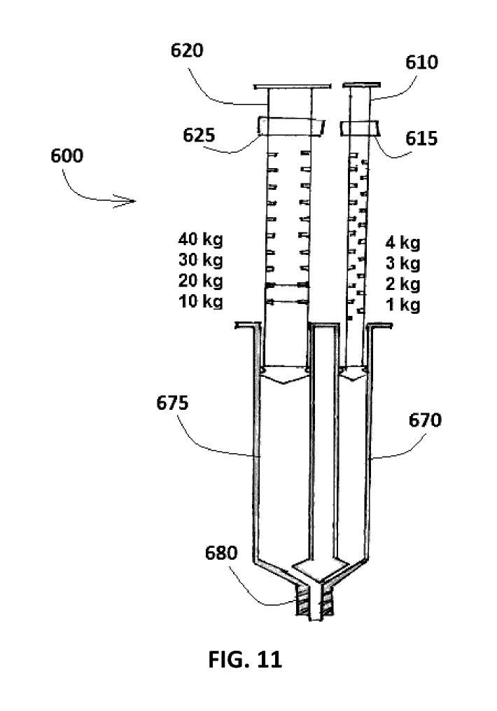

FIG. 11 includes a view of syringe 600 according to one example. Syringe 600 includes barrel 670 with plunger 610 (fine) and barrel 675 with plunger 620 (coarse) in parallel alignment. The barrel bore diameters differ and allow for a coarse selection and a fine selection. The corresponding barrels are marked to indicate, for example 40, 30, 20, and 10 kg and 4, 3, 2, and 1 kg dose. A single connector 680 (such as a Luer-lock fitting) is provided on the combination of barrels. Collars 615 and 625 are fitted to the plungers and are user positionable along a length of the respective plungers. In various examples, the collar positions are independently or dependently selectable.

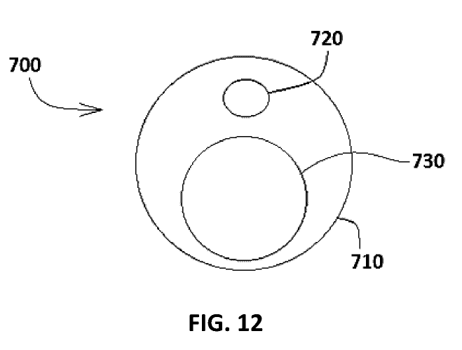

FIG. 12 includes a view of syringe 700 according to one example. Syringe 700 is shown along an axis of the barrel corresponding to FIG. 11. Barrel outer dimension 710 is indicated. The barrel includes two internal bores, including bore 720 (minor, or fine) and bore 730 (major, or coarse), however, any number of bores can be provided. The bores are eccentric relative to the outer diameter of the barrel and manipulation of the various plungers can deliver a selected dose.

The examples shown in FIGS. 11 and 12 allow good precision in selecting dosing. In addition, the increments of volume can be tailored based on the diameter of the various bores. In one example, an anti-back drive mechanism (or check-valve) is provided to preclude unintended movement of one plunger while actuating a second plunger. Spacing between the multiple caps can be tailored to accommodate ergonomic considerations.

Consider an example for a particular application. In this example, the barrel (or plunger) length is configured to provide a predetermined volume of medicament. For example, the rate for a particular syringe can be approximately 1 mm per kg dosing. A working length can be 80 kg (for a relatively large pediatric patient) and the total length can be about 20 cm. Improved precision in dosing can be achieved by providing a mechanical depth stop.

The dosing precision is a function of the dosing range for a particular syringe. If the range is 40 kg, then the syringe can be operated with twice the precision of a similar syringe having 80 kg range.

In one example, a kit includes a plurality of syringes having different dose ranges and different diameters or lengths.

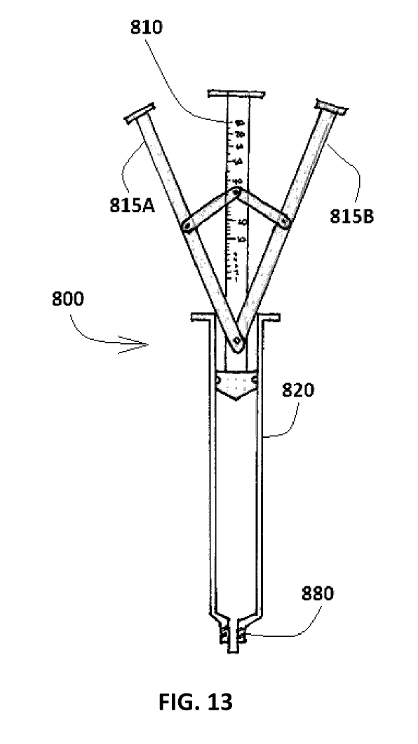

FIG. 13 includes a view of syringe 800 according to one example. Syringe 800 includes a single plunger 810 fitted to a single-bore barrel 820. Plunger 810 is fitted with adjustable actuators 815A and 815B. Gradations indicated on plunger 810 are configured to collapses to a typical syringe size (no ratchet mechanisms stick out). A discharge end includes fitting 880.

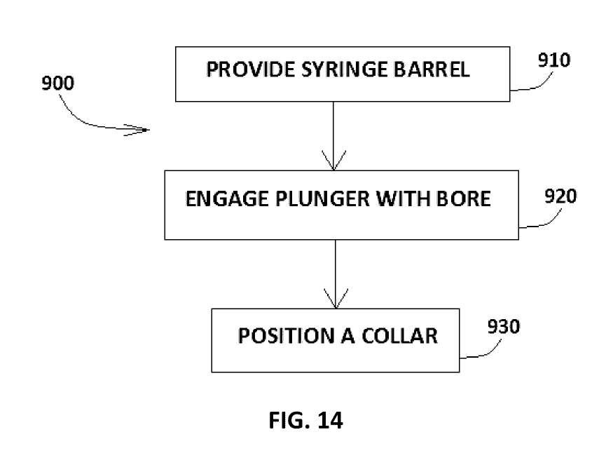

FIG. 14 includes a flow chart of method 900 according to one example. At 910, method 900 includes providing a syringe barrel. The syringe barrel can have a longitudinal axis aligned axially between a discharge end and a cylinder end.

At 920, method 900 includes engaging a syringe plunger with a bore of the syringe barrel. The plunger has a cap disposed on a first end, a piston disposed on a second end, and a body disposed between the first end and the second end. The body has an external thread on a first surface and the body has a visible calibration indication aligned axially.

At 930, method 900 includes positioning an annular collar on the body. The collar has at least one interrupted internal thread segment on an interior surface.

The collar has elasticity such that in a first state, the internal thread is configured to engage the external thread and in a second state, in which a compressive force is exerted on opposing sides of the collar, the internal thread is disengaged from the external thread.

Method 900 can include providing a calibration indication disposed in a recess of the body.

Method 900 can include providing a finger grip region on an opposing side of the collar.

### VARIOUS NOTES

The medicament volume gradations can be calibrated to correspond to patient weight using a color, a numeric value, or both a color and a number.

The barrel of the syringe can be configured to receive a Luer-lock fitting.

The collar and the barrel can be configured for use with an adult-sized auto-injector syringe and thus allow retrofit for use with a pediatric patient.

A syringe can have a barrel marked with calibration indicating patient weight values in numbers here representing kilograms.

The external threads on the plunger body can be continuous or discontinuous and, in one example, the threads are discontinuous and disposed on a face of the body. The discharge end of the barrel can include a discharge fitting, such as a Luer-lock connector.

The present subject matter can include a number of aspects, some of which follow:

Aspect 1 can include or use subject matter (such as an apparatus, a system, a device, a method, a means for performing acts), such as can include or use a syringe barrel, a syringe plunger, and an annular collar. The syringe barrel can include a longitudinal axis aligned axially between a discharge end and a cylinder end. The syringe plunger can include a cap disposed on a first end, a piston disposed on a second end and configured for disposition in a bore of the barrel, and can include a body disposed between the first end and the second end. The body can include an external thread on a first surface and the body can include a visible calibration indication aligned axially. The annular collar can include at least one interrupted internal thread segment on an interior surface. The collar can have elasticity such that in a first state, the internal thread is configured to engage the external thread and in a second state, in which a compressive force is exerted on opposing sides of the collar, the internal thread is disengaged from the external thread.

Aspect 2 can include or use, or can optionally be combined with the subject matter of Aspect 1, to optionally include or use wherein the external thread is continuous.

Aspect 3 can include or use, or can optionally be combined with the subject matter of one or any combination of Aspects 1 or 2 to optionally include or use wherein the external thread is discontinuous and disposed on a face of the body.

Aspect 4 can include or use, or can optionally be combined with the subject matter of one or any combination of Aspects 1 through 3 to optionally include or use wherein the calibration indication is disposed in a recess of the body.

Aspect 5 can include, or can optionally be combined with the subject matter of one or any combination of Aspects 1 through 4 to optionally include or use wherein the calibration indication includes a color coding associated with weight.

Aspect 6 can include, or can optionally be combined with the subject matter of one or any combination of Aspects 1 through 5 to optionally include or use wherein the calibration indication includes a coding associated with chronological age.

Aspect 7 can include, or can optionally be combined with the subject matter of one or any combination of Aspects 1 through 6 to optionally include or use wherein the collar includes a first contact face configured to engage a corresponding face proximate the cylinder end.

Aspect 8 can include, or can optionally be combined with the subject matter of one or any combination of Aspects 1 through 7 to optionally include or use wherein the collar includes a finger grip region on an opposing side.

Aspect 9 can include, or can optionally be combined with the subject matter of one or any combination of Aspects 1 through 8 to optionally include or use wherein the collar includes a plastic material.

Aspect 10 can include, or can optionally be combined with the subject matter of one or any combination of Aspects 1 through 9 to optionally include or use wherein the discharge end includes a discharge fitting.

Aspect 11 can include, or can optionally be combined with the subject matter of Aspects 10 to optionally include or use wherein the discharge fitting includes a Luer-lock connector.

Aspect 12 can include or use subject matter (such as an apparatus, a system, a device, a method, a means for performing acts), such as can include or use a syringe plunger and an annular collar. The syringe plunger can include a body disposed between a cap end and a piston end, the body having external threads and having visible gradations corresponding to a travel dimension of the plunger relative to a corresponding syringe barrel. The annular collar can include an interior surface and an exterior surface, the collar having elasticity, the interior surface having at least one segment of interrupted internal threads, and wherein in a first state, the internal threads are configured to engage the external threads, and in a second state, in which a compressive force is exerted on opposing sides of the exterior surface, the internal thread is disengaged from the external thread.

Aspect 13 can include, or can optionally be combined with the subject matter of Aspect 12 to optionally include or use wherein the at least one segment includes a first segment and a second segment aligned in diametric opposition.

Aspect 14 can include, or can optionally be combined with the subject matter of Aspects 13 to optionally include or use wherein the opposing sides are aligned normal to a line between the first segment and the second segment.

Aspect 15 can include, or can optionally be combined with the subject matter of one or any combination of Aspects 12 through 14 to optionally include or use wherein the external thread is continuous.

Aspect 16 can include, or can optionally be combined with the subject matter of one or any combination of Aspects 12 through 15 to optionally include or use wherein the external thread is discontinuous and disposed on a face of the body.

Aspect 17 can include, or can optionally be combined with the subject matter of one or any combination of Aspects 12 through 16 to optionally include or use wherein the gradations are disposed in a recess of the body.

Aspect 18 can include, or can optionally be combined with the subject matter of one or any combination of Aspects 12 through 17 to optionally include or use wherein the gradations include a color code associated with weight.

Aspect 19 can include, or can optionally be combined with the subject matter of one or any combination of Aspects 12 through 18 to optionally include or use wherein the gradations include a coding associated with chronological age.

Aspect 20 can include, or can optionally be combined with the subject matter of one or any combination of Aspects 12 through 19 to optionally include or use wherein the collar includes a first contact face configured to engage a corresponding face proximate a cylinder end of the syringe barrel.

Aspect 21 can include, or can optionally be combined with the subject matter of one or any combination of Aspects 12 through 20 to optionally include or use wherein the collar includes a finger grip region on an opposing side.

Aspect 22 can include, or can optionally be combined with the subject matter of one or any combination of Aspects 12 through 21 to optionally include or use wherein the collar includes a plastic material.

Aspect 23 can include or use subject matter (such as an apparatus, a system, a device, a method, a means for performing acts), such as can include or use providing a syringe barrel, engaging a syringe plunger with a bore of the syringe barrel, and positioning an annular collar on the body. Providing the syringe barrel entails, wherein the barrel has a longitudinal axis aligned axially between a discharge end and a cylinder end. Engaging the syringe plunger with the bore of the syringe barrel entails wherein the plunger has a cap disposed on a first end, a piston disposed on a second end, and has a body disposed between the first end and the second end, the body having an external thread on a first surface and the body having a visible calibration indication aligned axially. Positioning the annular collar on the body entails wherein the collar has at least one interrupted internal thread segment on an interior surface, the collar having elasticity such that in a first state, the internal thread is configured to engage the external thread and in a second state, in which a compressive force is exerted on opposing sides of the collar, the internal thread is disengaged from the external thread.

Aspect 24 can include, or can optionally be combined with the subject matter of Aspects 23 to optionally include or use providing a calibration indication disposed in a recess of the body.

Aspect 25 can include, or can optionally be combined with the subject matter of one or any combination of Aspects 23 through 24 to optionally include or use further including providing a finger grip region on an opposing side of the collar.

In addition to the foregoing aspects, the present subject matter can be explained by way of various examples, some of which are as follows:

Example 1 includes a device having a syringe barrel, a plunger, and a collar. The syringe barrel has an internal bore. The plunger is coupled to a piston and the piston is configured for axial movement in the bore. The collar is rotatably coupled to the barrel and rotation of the collar displaces a position of a depth stop relative to the internal bore. The position of the depth stop is associated with a volume in the internal bore. The volume is determined by the depth stop.

Example 2 includes the device of Example 1 and wherein the collar is coupled to the barrel by a thread.

Example 3 includes the device of Example 1 and wherein the collar is marked to indicate a weight.

Example 4 includes the device of Example 1 and wherein the collar is marked by a plurality of colors.

Example 5 includes the device of Example 1 and wherein the piston is coupled to a plunger and wherein the plunger is marked to indicate a weight.

Example 6 includes the device of Example 1 and wherein the piston is coupled to a plunger and wherein the plunger is marked to by a plurality of colors.

Example 7 includes the device of Example 1 and wherein the piston is coupled to a user-operable plunger.

Example 8 includes the device of Example 1 and wherein the user-operable plunger includes a cap and wherein the cap is configured to engage the depth stop.

Example 9 includes the device of Example 1 and wherein the collar is configured to have a first shape and a deformed shape.

Example 10 includes the device of Example 9 and wherein a thread of the collar, in the deformed shape, is disengaged from a corresponding thread of the barrel.

Example 11 includes device having a plurality of parallel bores, a plurality of pistons, and a calibration marking. The plurality of bores are coupled to a single discharge fitting. Each piston of the plurality of pistons is configured for axial movement within a bore. The pistons and bores are in one to one relation. The calibration marking is on a visible portion of a bore of the plurality of parallel bores or a plunger coupled to at least one piston of the plurality of pistons.

Example 12 includes the device of example 11 and wherein a position of a first piston of the plurality of pistons is independent of a position of a second piston of the plurality of pistons.

Example 13 includes the device of example 11 and wherein a diameter of a first piston of the plurality of pistons differs from a diameter of a second piston of the plurality of pistons.

Example 14 includes a device having a first barrel, a first piston, and a second piston. The first barrel has a first bore and a second bore. The first bore has a diameter different than a diameter of the second bore. The first piston is configured to slidably couple with the first bore. The first piston is coupled to a first cap by a first plunger. The first piston has an inner aperture. The second piston is slidably coupled to the second bore. The second piston is coupled to a second cap by a second plunger. The second plunger is slidably coupled to the aperture.

Example 15 includes the device of example 14 and wherein an axial position of the first piston and an axial position of the second piston are independently selectable.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A system comprising:
a syringe barrel having a longitudinal axis aligned axially between a discharge end and a cylinder end;
a syringe plunger having a cap disposed on a first end, a piston disposed on a second end and configured for disposition in a bore of the barrel, and having a body disposed between the first end and the second end, the body having an external thread on a first surface and the body having a visible calibration indication aligned axially; and
an annular collar having at least one interrupted internal thread segment on an interior surface, the collar having elasticity such that in a first state, the internal thread is configured to engage the external thread and in a second state, in which a compressive force is exerted on opposing sides of the collar, the internal thread is disengaged from the external thread.

2. The system of claim 1 wherein the external thread is discontinuous and disposed on a face of the body.

3. The system of claim 1 wherein the calibration indication is disposed in a recess of the body.

4. The system of claim 1 wherein the calibration indication includes a color coding associated with weight.

5. The system of claim 1 wherein the calibration indication includes a coding associated with chronological age.

6. The system of claim 1 wherein the collar includes a first contact face configured to engage a corresponding face proximate the cylinder end.

7. The system of claim 1 wherein the collar includes a finger grip region on an opposing side.

8. The system of claim 1 wherein the collar includes a plastic material.

9. A device comprising:
a syringe plunger having a body disposed between a cap end and a piston end, the body having external threads and having visible gradations corresponding to a travel dimension of the plunger relative to a corresponding syringe barrel, the piston end configured for movement in the barrel; and
an annular collar having an interior surface and an exterior surface, the collar having elasticity, the interior surface having at least one segment of interrupted internal threads, and wherein in a first state, the internal threads are configured to engage the external threads, and in a second state, in which a compressive force is exerted on opposing sides of the exterior surface, the internal thread is disengaged from the external thread.

10. The device of claim 9 wherein the at least one segment includes a first segment and a second segment aligned in diametric opposition.

11. The device of claim 10 wherein the opposing sides are aligned normal to a line between the first segment and the second segment.

12. The device of claim 9 wherein the gradations are disposed in a recess of the body.

13. The device of claim 9 wherein the gradations include a color code associated with weight.

14. The device of claim 9 wherein the gradations include a coding associated with chronological age.

15. The device of claim 9 wherein the collar includes a first contact face configured to engage a corresponding face proximate a cylinder end of the syringe barrel.

16. The device of claim 9 wherein the collar includes a finger grip region on an opposing side.

17. The device of claim 9 wherein the collar includes a plastic material.

18. A method comprising:
providing a syringe barrel, the barrel having a longitudinal axis aligned axially between a discharge end and a cylinder end;
engaging a syringe plunger with a bore of the syringe barrel, the plunger having a cap disposed on a first end, a piston disposed on a second end, and having a body disposed between the first end and the second end, the body having an external thread on a first surface and the body having a visible calibration indication aligned axially; and
positioning an annular collar on the body, the collar having at least one interrupted internal thread segment on an interior surface, the collar having elasticity such that in a first state, the internal thread is configured to engage the external thread and in a second state, in which a compressive force is exerted on opposing sides of the collar, the internal thread is disengaged from the external thread.

19. The method of claim 18 further including providing a calibration indication disposed in a recess of the body.

20. The method of claim 18 further including providing a finger grip region on an opposing side of the collar.
